SEQUENCE LISTING (14 sequences)

SEQ ID NO: 1 - DNA, 24 bp, Artificial Sequence
  TD1F
gaccagcatttcgtttccattgga

SEQ ID NO: 2 - DNA, 24 bp, Artificial Sequence
  TDR1R
aggaatggaaaacaaatgctgcag

SEQ ID NO: 3 - DNA, 21 bp, Artificial Sequence
  TD3F
gtcaaatgggtgtttccaaga

SEQ ID NO: 4 - DNA, 21 bp, Artificial Sequence
  TD3R
acttggaatctcccatttgag

SEQ ID NO: 5 - DNA, 21 bp, Artificial Sequence
  TD4F
catctctaaagggagtttcca

SEQ ID NO: 6 - DNA, 21 bp, Artificial Sequence
  TD4R
aggaaactcccaaatgagatc

SEQ ID NO: 7 - DNA, 25 bp, Artificial Sequence
  TD5F
ctcatcttcttcgttgatttcagag

SEQ ID NO: 8 - DNA, 25 bp, Artificial Sequence
  TD5R
gagacaaatctacgtagaagtactc

SEQ ID NO: 9 - DNA, 26 bp, Artificial Sequence
  TD6F
gagtcataatgtgtacttctacgttg

SEQ ID NO: 10 - DNA, 26 bp, Artificial Sequence
  TD6R
gttgctagaagaactcattatctgag

SEQ ID NO: 11 - DNA, 23 bp, Artificial Sequence
  TD7F
actccggctcctctgataatgag

SEQ ID NO: 12 - DNA, 23 bp, Artificial Sequence
  TD7R
gagattatctgtggagccggtca

SEQ ID NO: 13 - DNA, 21 bp, Artificial Sequence
  TD9F
gagctacagttggtacaggtg

SEQ ID NO: 14 - DNA, 21 bp, Artificial Sequence
  TD9R
gacctgtaccttctgtagctg

(19) United States
(12) Patent Application Publication
(10) Pub. No.: US 2015/0275310 A1
(43) Pub. Date: Oct. 1, 2015

(54) METHODS AND COMPOSITIONS FOR DETECTING INTERNAL TANDEM DUPLICATION MUTATIONS

(71) Applicant: THE JOHNS HOPKINS UNIVERSITY, Baltimore, MD (US)

(72) Inventors: Ming-Tseh Lin, Clarksville, MD (US); Christopher Gocke, Ellicott City, MD (US)

(21) Appl. No.: 14/439,352

(22) PCT Filed: Nov. 4, 2013
(86) PCT No.: PCT/US13/68193

§ 371 Date: Apr. 29, 2015

Related U.S. Application Data
(60) Provisional application No. 61/721,708, filed on Nov. 2, 2012.

(51) Int. Cl. C12Q 1/68

(52) U.S. Cl. CPC C12Q 1/6886; C12Q 2600/158; C12Q 2600/156

(57) ABSTRACT

The present invention relates to the field of diagnostics methods and compositions. More specifically, the present invention provides methods for detecting internal tandem duplication (ITD) mutations. In a specific embodiment, a method for detecting an internal tandem duplication in the genome of a cell comprising the steps of (a) providing a DNA sample isolated from the cell; (b) contacting the DNA sample with a forward primer that hybridizes within the tandem duplicated sequence; (c) contacting the DNA sample with a reverse primer upstream of the forward primer that hybridizes within the tandem duplicated sequence; (d) amplifying the region between the two primers; and (e) identifying the cell as containing an internal tandem duplication if an amplification product of the expected size is detected.

### CROSS-REFERENCE TO RELATED APPLICATIONS

This application claims the benefit of U.S. Provisional Application No. 61/721,708 filed Nov. 2, 2012, which is incorporated herein by reference in its entirety.

### STATEMENT OF GOVERNMENTAL INTEREST

This invention was made with government support under grant no. HG005745 awarded by the National Institutes of Health. The government has certain rights in the invention.

### INCORPORATION-BY-REFERENCE OF MATERIAL SUBMITTED ELECTRONICALLY

This application contains a sequence listing. It has been submitted electronically via EFS-Web as an ASCII text file entitled “P12085-02_ST25.txt.” The sequence listing is 2,750 bytes in size, and was created on Nov. 4, 2013. It is hereby incorporated by reference in its entirety.

### FIELD OF THE INVENTION

The present invention relates to the field of diagnostics methods and compositions. More specifically, the present invention provides methods for detecting internal tandem duplication (ITD) mutations.

### BACKGROUND OF THE INVENTION

FMS-like tyrosine kinase (FLT3), a member of the class III receptor tyrosine kinase family (1), plays a key role in survival of hematopoietic progenitor cells (2). Internal tandem duplication (ITD) mutations of the FLT3 gene occur in 20-30% of acute myeloid leukemia (AML) and have been associated with an inferior prognosis (3-6). Currently, most AML patients with ITD mutations need hematopoietic stem cell transplantation. As a corollary, identification of FLT3 mutations has become a routine assay to provide optimal treatment for AML patients. ITD mutations also provide a potentially useful molecular marker for monitoring minimal residual disease (MRD) (7, 8).

ITD mutations of the FLT3 gene typically result from head-to-tail insertion of a duplicated portion of the juxtamembrane region (FIG. 1) (9). A standard PCR assay using primers that straddle the ITD mutation has been developed to detect length-altering mutations of ITD (10). Amplicons with a size greater than that of wild-type and labeled with both 6-FAM and HEX are interpreted as positive for ITD mutation. The limit of detection is generally reported as about 10% leukemia cells (5% mutant alleles) since amplicons with low peak heights, even though labeled with both fluorochromes, may represent non-specific amplification.

The present inventors recently developed a triple-primer strategy, delta-PCR, to ensure PCR specificity and improve the limit of detection to 0.1% leukemia cells (11). The present inventors demonstrated that minor leukemia mutants with an ITD mutant allelic burden of less than 1% detected by delta-PCR at the initial diagnosis could be clinically significant. An assay with even higher sensitivity, however, is needed for monitoring MRD. However, both standard PCR assays and our delta-PCR approach are intrinsically flawed for detecting ITD: the amplicon from the wild-type allele is always shorter than the mutant allele and thus favored in amplification (FIG. 1). Attempts have been made to reduce this intrinsic bias by reducing PCR cycle number, but this of course reduces the ability to detect rare mutants, i.e., MRD (5).

### SUMMARY OF THE INVENTION

The present invention is based, at least in part, on the development of an assay to identify internal tandem duplication mutations in a given region of the genome using a unique arrangement of DNA oligonucleotides that preferentially amplify the duplicated segment. Internal tandem duplications are mutations, generally found in cancer but also in other conditions, which result in a head-to-tail joining of a duplicated stretch of DNA. They are important, particularly in leukemia (20% of cases with FLT3 gene), but also in many other genes in many other cancers. Although the work described herein was performed in the context of cancer for detecting internal tandem duplication, the present invention is not so limited. Thus, the methods and compositions of the present invention are applicable to the detection of tandem duplications in a variety of diseases or conditions.

Current art straddles the duplicated segment with oligonucleotides, amplifies them by PCR, and performs a sizing assay to identify mutants that yield larger amplicons than the normal, which is also amplified by straddling primers. The present invention, Tandem Duplication PCR (TD-PCR), relies on a unique arrangement and sequence of the primers to amplify ITDs. By overlapping the primers and pointing them in opposing directions, only duplicated segments of DNA will be amplified. This reduces background amplification from normal DNA to zero. What was formerly a favored amplicon (normal) because it was the shortest product in a PCR reaction is no longer a factor, dramatically increasing the analytic sensitivity of an assay for the ITD. In particular, the present invention provides the ability to do minimal disease detection to a single cell level, something not obtainable with current technology.

Although TD-PCR is described in detail as applied to the FLT3 gene mutation, the present invention is not to be construed as being so limited. Indeed, the present invention is applicable to any disease associated with an internal tandem duplication mutation including, but not limited to, other common oncogenes such as EGFR (lung and colon cancer), KIT (leukemia and melanoma) and p53 (various cancers). Pre-malignant conditions, such as adenomas, and diseases other than cancer may also have unique tandem duplication changes in the genome amenable to detection. What changes is the primer sequence; the approach and specific methods are the same.

As described here, TD-PCR is an ultra-sensitive assay for the detection of tandem duplications. In standard PCR for FLT3/ITD mutations (FIG. 1), template specific products are amplified from both the smaller wild-type allele and the larger mutant allele, resulting in competition for amplification and reduced mutant signal. The reaction in TD-PCR (FIG. 1), however, includes an upstream reverse primer (R1) and a downstream forward primer (F1), instead of an upstream forward primer (F) and a downstream reverse primer (R) for standard PCR. When both TD-PCR primers are located within the tandem duplicated sequences, amplification occurs across the insertion junction using the forward primer located within the upstream duplicated sequence and the reverse primer located within the downstream duplicated sequence. No template-specific amplification occurs from the wild-type allele or if one or both primers lie outside the duplicated segment.

### BRIEF DESCRIPTION OF THE FIGURES

FIG. 1: Design for TD-PCR. In standard PCR for detecting ITDs, template-specific PCR products are amplified by forward primer F and reverse primer R from both wild-type (top) and mutant (bottom) alleles with preferential amplification of the smaller sized wild-type allele. In the TD-PCR, template-specific PCR products are only amplified from the mutant allele by forward primer F1 and reverse external and internal primers R1 and R2. Either or both of the reverse primers may be employed. “Δ” (delta) indicates the defined size difference of the paired amplicons (19±1 bases in this study). “→” indicates primers.

FIG. 2: Design of TD-PCR according to duplication sequences of an ITD mutant undetectable by both delta-PCR (13) and standard PCR (10). The ITD fragment extends from exon 14 to near the 3′end of intron 14 (A). A pair of amplicons migrating at 141 bases and 161 bases was detected by TD-PCR using forward primer INT14F4 (F4) and a pair of reverse primers, external INT14R1 (R1) primer and internal INT14R2 (R2) primer (B). The internal primer served as a “probe” to ensure PCR specificity. A pair of amplicons with a delta (“Δ”) of 19±1 bases confirms an ITD mutation (C). The black peak in (C) is an internal control amplicon unrelated to the ITD. Direct sequencing of the 161-base amplicon confirmed the abnormal junction (vertical arrow) between intron 14 and exon 14 (D). Red box indicates the duplicated part of exon 14 and the red line indicates the duplicated part of intron 14. P14F and P15R are the primers used for standard PCR for detection of ITD mutations (10). RFU: relative fluorescence units.

FIG. 3: Limit of detection of TD-PCR. DNA samples from case A115 with ITD mutation were serially diluted with normal DNA samples (1 in 2,000 in 3A, 1 in 10,000 in 3B, 1 in 20,000 in 3C and negative control in 3D). “Δ” indicates the defined size difference of the paired amplicons at 19±1 bases. The 150-base NED-labeled black peak is an internal control amplicon for PCR. RFU: relative fluorescence unit.

FIG. 4: TD-PCR was used to detect ITD mutations which were undetectable by standard PCR. Delta-PCR showed a pairs of amplicons with extremely low peak heights at 90-second injection in case A185 (A). A pair of amplicons with peak heights of several thousands at 25-second injection was detected by TD-PCR in case A185 (B) and case A23 (C). “Δ” indicates the defined size difference of the paired amplicons at 19±1 bases. The 150-base NED-labeled black peak is an internal control amplicon for PCR. RFU: relative fluorescence unit.

FIG. 5: Revision of TD-PCR. The forward primer (→) is located downstream, instead of upstream in standard PCR, of the reverse primer (←) (5A and 5B). Primer span is defined as the length between the 3′ ends of the reverse and forward primers (5A). Template-specific PCR products are only amplified from the mutant allele by the forward primer of pair 2 within the upstream duplication segment and the reverse primer of pair 2 within the downstream duplication segment (5B). In the revised design, forward and reverse primers are the complementary sequences of each other to reduce the primer span (number in the parenthesis) (5C). Mismatched nucleotides (underlined) were introduced at the 5′ end and the middle portion of the primers to reduce annealing between primers (5D). In this primer pair (TD7), mismatches between the forward and reverse primers were designed in 8 (labeled with *) of 23 nucleotides including 3 mismatched nucleotides at the 3′ end of each primer.

FIG. 6A-6F: Template specific amplification of ITD by revised TD-PCR. Amplification by two or more adjacent primer pairs with amplicons of the same size or 1-5 bases difference depending on the primer span of the primer pairs indicates duplication specific amplification from an ITD mutant. PS: primer span (21-26 bases). TD-PCR was conducted in a total of 2 ng DNA. TD-PCR products were diluted 100 fold before electrophoresis. Number in the parenthesis indicates the observed amplicon size determined by capillary electrophoresis. RFU: relative fluorescence unit. Arrows indicate amplicons from the same ITD mutant.

FIG. 7A-7F: Limit of detection of revised TD-PCR for primer pairs TD3. DNA samples from a diagnostic FLT3/ITD AML of nearly 100% leukemia cells were serially diluted with a normal DNA sample: 1 in 1,000 (10⁻³ in F), 1 in 2,000 (5×10⁻⁴ in E), 1 in 10,000 (10⁻⁴ in D), 1 in 20,000 (5×10⁻⁵ in C) and 1 in 100,000 (10⁻⁵ in B). PCR products from 1:1,000 and 1:2,000 diluents were diluted for 10 fold before electrophoresis. The red vertical lines indicate off-scale signal.

FIG. 8: Event-free survival for patients positive versus negative by TD-PCR at day 60 following hematopoietic cell transplantation.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

FLT3/ITD mutations are present in approximately one quarter of adult AML cases. Patients with these mutations carry a particularly poor prognosis. For this reason, a number of small molecule FLT3 inhibitors are being evaluated in clinical trials. In AML patients with FLT3/ITD mutations, it has become an increasingly common practice to pursue allogeneic bone marrow transplantation quickly during first remission. Among this patient population, as with other AML patients, relapse following transplant occurs frequently. As a result, there are numerous ongoing efforts to investigate a potential role for maintenance therapy with FLT3 inhibitors in the post-transplant setting. Historically, post-transplant remission in AML patients has been assessed using bone marrow morphology as well as hematologic recovery. Better methods are needed to assess minimal residual disease in AML patients. The standard method of detecting the FLT3/ITD mutation (10) with PCR has a limit of detection of approximately 1 in 10 cells. In many FLT3/ITD AML patients who ultimately relapse with FLT3-mutated disease after transplant, the FLT3 mutation is still not detectable by standard PCR assay at time points before and after transplant, because their burden of disease is lower than the detection threshold of the assay.

The present inventors describe herein a new PCR technique for detecting the presence of ITD mutations with a high level of sensitivity and specificity. The technique is referred to as tandem duplication PCR (TD-PCR). Instead of using inward-facing primers that flank the juxtamembrane domain of FLT3, TD-PCR uses outward-facing, overlapping primers within the expected region of mutation. The TD-PCR primers will not amplify any product when annealed to a wild type FLT3 template. However, if a tandem duplication is present, the primers will anneal at two distinct sites within the coding sequence and will amplify a product.

In certain embodiments, the present invention provides methods for detecting an internal tandem duplication in the genome of a cell comprising the steps of (a) providing a DNA sample isolated from the cell; (b) contacting the DNA sample with a forward primer that hybridizes within the tandem duplicated sequence; (c) contacting the DNA sample with a reverse primer upstream of the forward primer that hybridizes within the tandem duplicated sequence; (d) amplifying the region between the two primers; and (e) identifying the cell as containing an internal tandem duplication if an amplification product of the expected size is detected. In other embodiments, the methods for detecting an internal tandem duplication in the genome of a cell further comprise contacting the DNA sample with a second reverse primer upstream of the first reverse primer, wherein the amplification step produces two amplification products of two different sizes. In other embodiments, the DNA may be isolated from extracellular fluids of a human or animal, such as plasma or serum, rather than from intact cells. This identifies the fluid as containing an internal tandem duplication if an amplification product is detected.

In other embodiments, a method for detecting an internal tandem duplication in the genome of a cell comprises the steps of (a) providing a DNA sample isolated from the cell; (b) contacting the DNA sample with a first primer set comprising: (i) a forward primer that hybridizes within the tandem duplicated sequence; (ii) a reverse primer upstream of the forward primer that hybridizes within the tandem duplicated sequence; (c) amplifying the region between the two primers; and (d) identifying the cell as containing an internal tandem duplication if an amplification product of the expected size is detected.

In another embodiment, the first primer set further comprises a second reverse primer upstream of the first reverse primer, wherein the amplification step produces two amplification products of two different sizes. In yet another embodiment, the method can further comprise contacting the DNA sample with a second primer set comprising primers that hybridizes to a different portion of the tandem duplicated sequence. In a further embodiment, a series of primer sets are arrayed across the DNA target, either overlapping or non-overlapping, and amplification from more than one primer set is confirmatory of an ITD. Such multiple amplifications may be used to size the ITD. In a further embodiment, the method can also comprise contacting the DNA sample with a second primer set comprising primers that hybridizes to a sequence of the tandem duplicated sequence that overlaps with the first primer set.

In other embodiments, a method for detecting an internal tandem duplication in the genome of a cell comprises the step of performing a polymerase chain reaction on DNA isolated from the cell using outward facing primers that hybridize within the tandem duplicated sequence.

In yet further embodiments, the present invention provides methods for detecting an internal tandem duplication in a sample from a subject. In one embodiment, the method comprises the steps of (a) providing a sample obtained from a subject; (b) contacting the sample with a forward primer that hybridizes within the tandem duplicated sequence; (c) contacting the sample with a reverse primer upstream of the forward primer that hybridizes within the tandem duplicated sequence; (d) amplifying the region between the two primers; and (e) identifying the sample as containing an internal tandem duplication if an amplification product of the expected size is detected. In a specific embodiment, the method further comprises contacting the sample with a second reverse primer upstream of the first reverse primer, wherein the amplification step produces two amplification products of two different sizes. In certain embodiments, the sample is a biological sample. In more specific embodiments, the sample is a blood, plasma or serum sample. The sample can further comprise other biological fluids including, but not limited to, urine, cerebrospinal fluid, amniotic fluid and the like. In particular embodiments, the subject is a human.

The present invention also provides kits for detecting internal tandem duplications. In one embodiment, a kit for detecting internal tandem duplications comprises (a) a forward primer that hybridizes with a tandem duplicated sequence; (b) a reverse primer that binds upstream of the forward primer that hybridizes within the tandem duplicated sequence; and (c) reagents for conducting an amplification reaction. In a specific embodiment, the kit further comprises a second reverse primer that binds upstream of the first reverse primer of element (b) above.

In certain embodiments, to improve the sensitivity and specificity of this assay, the TD-PCR reaction can be carried out using several pairs of primers, each slightly shifted within the target sequence in comparison to the others. In specific embodiments, the present inventors have designed seven pairs of TD-PCR primers located within exon 14 and intron 14 to successfully amplify approximately 70% of ITD mutants. The ability to detect the presence of just a single molecule of a great majority of mutants anywhere within the target sequence can be reached by using several primer pairs. The present inventors estimate that when about 5 ug of DNA from a good quality bone marrow aspirate is analyzed under optimum conditions using this method, the sensitivity is such that a single ITD mutant cell can be detected out of 100,000 normal cells. This is in the range of the BCR-ABL PCR assays currently in widespread clinical use. ITD mutants with duplication of less than 25-30 bases cannot be amplified by this assay. However, ˜70% of FLT3/ITD patients presenting at the inventors' institution over the past 10 years have had ITD mutations of sufficient length to detect in this assay.

The assay has been used to detect the persistent ITD mutant in the bone marrow of a patient 2 months after bone marrow transplantion, which the standard assay failed to detect. This patient had clinical relapse 6 months post-transplant. DNA can be extracted using the QIAamp DNA kit (Qiagen, Valencia, Calif.) according to the manufacturer's instructions. For each patient, 1-5 ug of DNA was used, in 8 individual PCR reactions using the tandem primer pairs. TD-PCR is conducted using a forward primer and a reverse primer labeled with 6-FAM. TD-PCR can be performed in a 100 μL volume containing 1 μg DNA, 50 pmol of each forward primer and reverse primer, 1.5 mM MgCl2, 0.2 mM each deoxyribonucleotide, 5 units AmpliTaq Gold DNA polymerase and 10 μL buffer (Applied Biosystems, Foster City, Calif.). Samples are subjected to 40 cycles of denaturation (95° C., 30 sec), annealing (57° C., 30 sec) and extension (72° C., 60 sec). In certain embodiments, to carry out this assay clinically, about 5 to about 10 ug of DNA from marrow is needed. In a single cell, there is ˜6×10⁻¹² grams (6 picograms) of DNA, so 6 ug corresponds to 1 million cells. Therefore, about 1 million cells are needed. A typical 5 mL sample of bone marrow aspirate in a green or purple top vacutainer tube will yield anywhere from 1 to 20 million cells when collected from a post-transplant patient. The tubes can be sent to a central location where the DNA can be prepared according to the standard protocol. In other embodiments, fewer cells may be used, but the limit of detection will not be as good.

Thus, the present invention provides a generic assay for tandem duplication detection with greatly improved sensitivity and specificity, to a limit of detection of a single molecule of an ITD. This assay can be applied to monitor MRD.

Without further elaboration, it is believed that one skilled in the art, using the preceding description, can utilize the present invention to the fullest extent. The following examples are illustrative only, and not limiting of the remainder of the disclosure in any way whatsoever.

### Examples

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Materials and Methods

Design of Tandem Duplication PCR (TD-PCR).

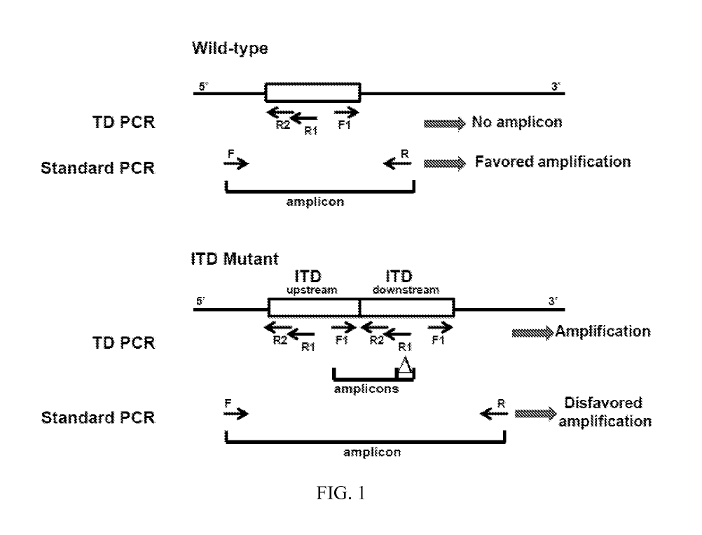

Inverse PCR is generally conducted with restriction enzyme digested DNA that is circularized by ligation, which allows adjacent PCR primers that face away from each other to amplify a product across the restriction site. There is no digestion or ligation; the arrangement of tandem duplicates permits PCR primers facing away from each other to produce an amplicon across the duplication junction. The reaction in TD-PCR includes an upstream reverse primer and a downstream forward primer, in contrast to an upstream forward primer and a downstream reverse primer for standard PCR (FIG. 1). When both primers are located within the tandem duplicated sequences, amplification occurs across the insertion junction using the forward primer located within the upstream duplicated sequence and the reverse primer located within the downstream duplicated sequence. Since the length-altering mutation varies for each ITD mutant, a third primer can also be added to the reaction to improve the specificity of the reaction, analogous to our delta-PCR (13). No template-specific amplification occurs from the wild-type allele or if one or both primers lie outside the duplicated segment. By contrast, in standard PCR, template specific products are amplified from both the smaller wild-type allele and the larger mutant allele, resulting in competition for amplification and reduced mutant signal (FIG. 1).

Polymerase Chain Reaction (Standard PCR, Delta-PCR and TD-PCR).

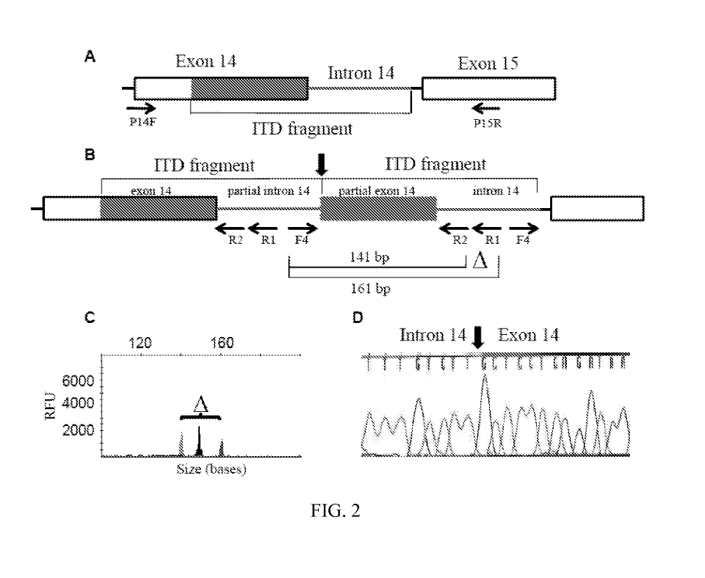

The standard PCR for simultaneously detecting an ITD mutation and D835 point mutation was performed as described (10). Amplicons with a size greater than that of wild-type (328±1 bases on ABI 3130×L capillary electrophoresis using the previously described oligonucleotides, FIG. 2a) and which were labeled with both 6-FAM and HEX fluorochromes were interpreted as positive for ITD mutation. Delta-PCR assay was performed using a forward primer and 2 reverse primers (external primer labeled with 6-FAM and internal primer labeled with HEX) as described previously (11). The presence of a pair of amplicons with a delta of 19±1 bases and with sizes larger than the pair of amplicons from the wild-type allele (160-base green peak and 179-base blue peak) was interpreted as positive for ITD mutation.

TD-PCR was conducted using a forward primer located at the 3′end of intron 14 of the FLT3 gene (primer INT14F4: 5′-aat gca cgt act cac cat ttg tc-3′) and a reverse primer located at the middle portion of intron 14 (primer INT14R1: 5′-caa tgg aaa aga aat get gca g-3′, labeled with 6-FAM) (FIG. 2b). The additional “internal” reverse primer (primer INT14R2: 5′-cag aaa cat ttg gca cat tcc a-3′, labeled with HEX) was located at the 5′end of intron 14. These two reverse primers are the same as those used for delta-PCR (11). As described previously for delta-PCR (11-13), the internal primer was used as a probe to ensure PCR specificity. For both delta-PCR and the new TD-PCR, a pair of amplicons with a delta of 19±1 bases was interpreted as positive for ITD mutation.

TD-PCR was performed in a 20 μL volume containing up to 250 ng DNA, 10 pmol of forward primer, 5 pmol of reverse external primer, 5 pmol of reverse internal primer, 1.5 mM MgCl₂, 0.2 mM each deoxyribonucleotide, 1.0 units AmpliTaq Gold DNA polymerase and 2 μL buffer (Applied Biosystems, Foster City, Calif.). A PCR quality control reaction (to the activation loop of FLT3) was included in every run (10). Samples were subjected to 40 cycles of denaturation (95° C., 30 sec), annealing (57° C., 30 sec) and extension (72° C., 60 sec).

Materials.

DNA was isolated from the peripheral blood or bone marrow of 117 consecutive AML patients at initial diagnosis or relapse using the QIAamp DNA Blood Mini Kit (Qiagen, Valencia, Calif.). Only cases reported to contain greater than 20% blasts in the sample were included. Detection of the ITD using standard PCR had been previously performed: 40 were ITD-positive and 77 were ITD-negative. An additional 6 ITD-positive AML patients with a larger duplication (more than 480 bases as compared to the wild-type peak migrating at 328±1 bases) were also selected for testing by TD-PCR. The Johns Hopkins Medicine institutional review board granted approval to this study.

Capillary Electrophoresis.

One μL PCR products, 0.5 μL ROX size standard and 8.5 μL deionized formamide were mixed according to the manufacturer's protocol (Applied Biosystems), heated at 95° C. for 2 minutes and placed on ice for at least 1 minute before electrokinetic injection (10 and 25 sec for TD-PCR) on the ABI 3130×L Genetic Analyzer (Applied Biosystems) as described previously (14). The size and positions of peaks on the electropherogram were analyzed using GeneMapper® analysis software (Applied Biosystems).

DNA Sequencing.

Primers INT14R1M13R and INT14F4M13F were their parent primers tagged with M13 sequences for direct sequencing of amplicons. PCR products were purified using USB ExoSapit (GE Healthcare, Uppsala, Sweden) and cycle sequenced using the BigDye Terminator version 3.1 cycle sequencing kit according to the manufacturer's protocol and resolved on an ABI 3500×L sequencer (Applied Biosystems). Sequences were analyzed using Sequencher software (Gene Codes Corp, Inc., Ann Arbor, Mich.).

### Results

Provided below are comparative results for three assays (standard PCR, delta-PCR and TD-PCR) in the setting of FLT3 ITD and the benefits of TD-PCR shown.

Rationale for Designing TD-PCR.

The present inventors previously reported the delta-PCR assay to detect minor ITD-positive mutants representing as little as approximately 0.1% of all cells. However, this is insufficient to measure true minimal residual leukemia. An assay with higher sensitivity was needed. TD-PCR was initially designed to confirm an ITD mutant which was undetectable by standard PCR or delta-PCR. This mutant, along with other 4 mutants detectable by delta-PCR, was isolated incidentally by a molecular fraction collecting tool (13, 14). A pair of amplicons, demonstrating the expected delta of 19±1 bases and migrating at 141 bases and 161 bases, was repeatedly detected by TD-PCR in the bone marrow specimen in which this mutant was isolated (FIG. 2c), but not the concurrent peripheral blood specimen (data not shown), suggesting that this minor clone was present in the bone marrow at a very low level. Sanger sequencing of the TD-PCR amplicons confirmed the presence of the same ITD sequences (c.1748_c.1838-5dup) identified from the amplicons isolated by our molecular fraction collection tool (FIG. 2d).

Limit of Detection of TD-PCR.

In a previous study, the present inventors demonstrated that a minor mutant with an ITD mutation may be clinically significant (11). In a patient with at least 3 ITD-positive mutants at initial diagnosis, a minor mutant with an allelic burden of 0.5%, which migrated at a position slightly more than 500 bases and was barely detected by standard PCR and delta-PCR, became the only mutant, with an allelic burden of 44% at the time of relapse. By contrast, in the initial diagnosis sample TD-PCR easily detected this minor mutant with a peak height over 100-fold higher than standard PCR (data not shown). Sanger sequencing confirmed the presence of an ITD of 186 bases extending from exon 14 into exon 15 (c.1754_c.1840 dup) that is consistent with the ITD mutant detected by standard PCR and delta-PCR.

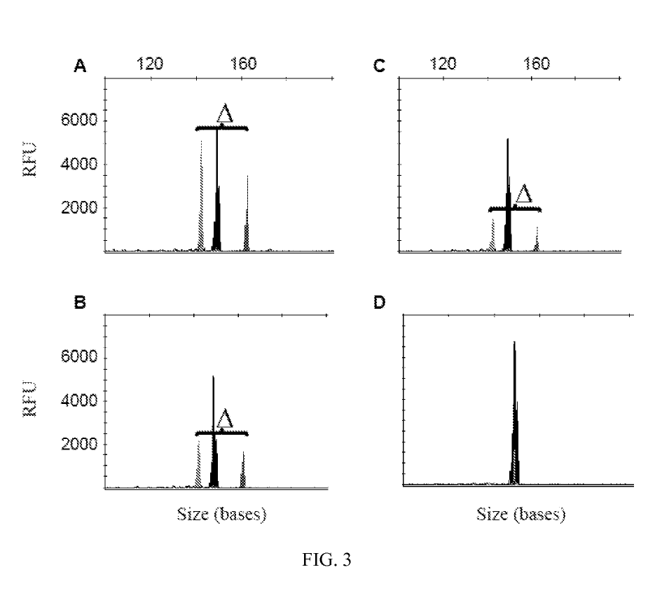

The relapsed specimen of this patient was serially diluted with an ITD negative specimen at 1:1,000 (10⁻³), 1:2,000 (5×10⁻⁴), 1:10,000 (10⁻⁴), and 1:20,000 (5×10⁻⁵). TD-PCR was conducted in replicates with 250 ng DNA (equivalent to approximately 40,000 cells) in each reaction. The expected pair of amplicons was detected in 4 of 4 replicates at a dilution of 10⁻³, 8 of 8 replicates at a dilution of 5×10⁻⁴ (equivalent to 20 mutant cells), 6 of 8 replicates at a dilution of 10⁻⁴ (equivalent to 4 mutant cells), and 5 of 8 replicates at a dilution of 5×10⁻⁵ (equivalent to 2 mutant cells), assuming all the leukemia cells contain this ITD mutant and each cell contains 6-7 pg DNA (FIG. 3). TD-PCR was also tested with a reduced amount of DNA (50 ng) in each reaction. The expected product was also detected in 15/16 replicates at a dilution of 5×10⁻⁴ (equivalent to 4 mutant cells) and in 2 of 16 replicates at a dilution of 10⁻⁴ (equivalent to 0.8 mutant cells). The results indicate that TD-PCR is a very sensitive assay for ITD, likely capable of detecting less than 5 mutant copies per reaction. In all cases, background was negligible.

Detection of ITD by TD-PCR in Patients with AML.

TD-PCR was used to detect ITD in 117 cases of newly diagnosed AML or frank relapsed AML. The assay was positive in 8 of 40 cases previously diagnosed as ITD-positive AML by standard PCR. Sanger sequencing confirmed the presence of ITD with duplications extending from exon 14 into intron 14 or exon 15 of the FLT3 gene and confirms that the current design of primers detected only ITD of larger size with duplication extending from exon 14 into intron 14. The cases not detected by TD-PCR had smaller ITDs by standard PCR and would not be expected to yield a positive result. An additional 6 selected cases with duplication sequences of more than 150 bases were also positive. Again, the duplication sequences were confirmed by Sanger sequencing of the amplicons from TD-PCR.

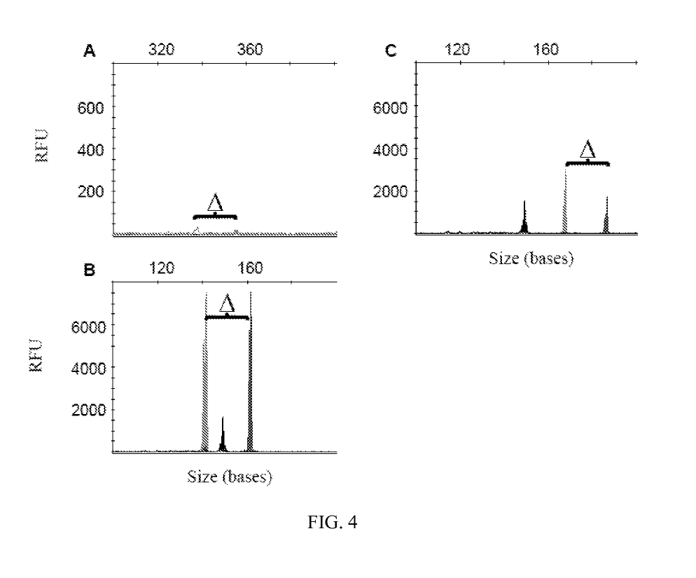

As a test of the clinical sensitivity of TD-PCR, 77 cases of AML that were previously reported as negative for ITD mutation by standard PCR were assayed. TD-PCR was conducted using 250 ng DNA. Two cases missed by standard PCR (cases A23 and A185) were identified as mutated by TD-PCR. The presence of ITD sequences was confirmed by Sanger sequencing in both cases. In case A185, a pair of amplicons with peak height less than 50 relative fluorescence units (RFU) (90 second injection) which was not easy to distinguish from background noise was observed in 2 of 5 reactions by delta-PCR (FIG. 4a). However, TD-PCR can easily detect this ITD mutation with a pair of peaks of more than 7,000 RFU (25 second injection) (FIG. 4b). TD-PCR consistently shows this ITD mutant even with as little as 50 ng input DNA. In case A23, a pair of amplicons was detected in TD-PCR reactions (FIG. 4c), but not in the corresponding delta-PCR reactions. This mutant was seen by TD-PCR in 6 of 16 additional replicates containing 50 ng DNA, suggesting a very low level of ITD mutant in case A23.

Reducing Primer Span to Increase TD-PCR Capability.

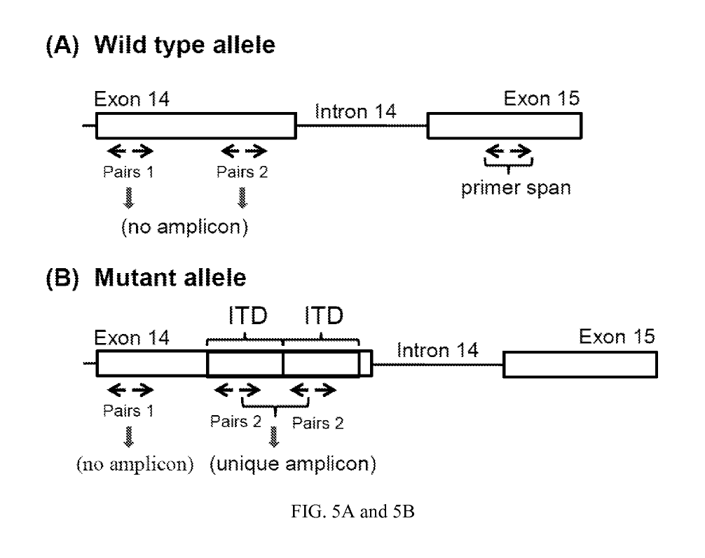

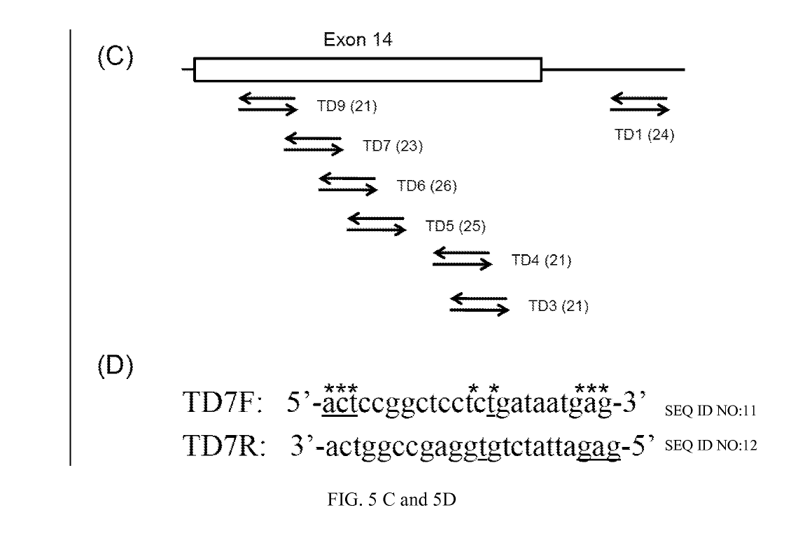

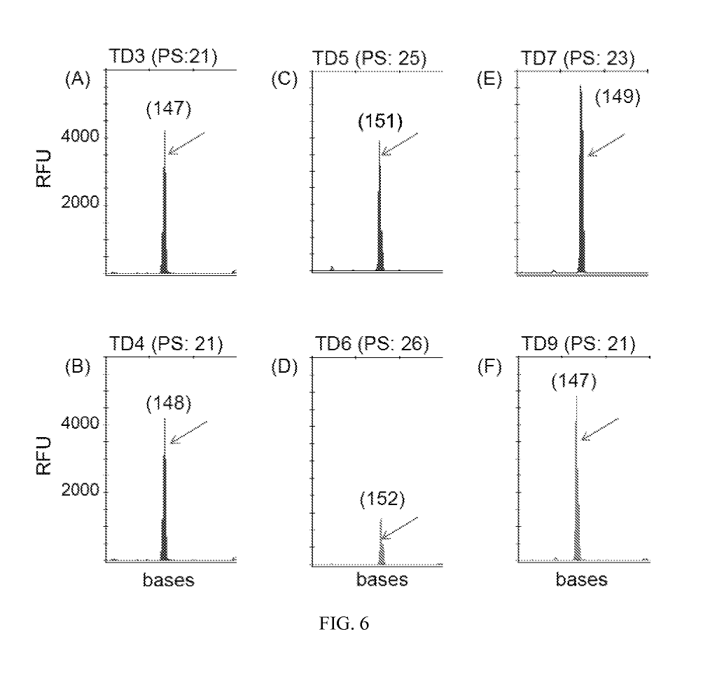

In order to improve the capability of TD-PCR for MRD detection of broader FLT3/ITD AMLs, the present inventors reduced the primer span, defined as the distance from the 3′ end of the reverse primer to the 3′ end of the forward primer (FIG. 5A). Because both primers need to lay within the duplication segment to amplify the ITD mutants by TD-PCR, the shorter the primer span the better. In the current design, the forward and reverse primers were the anti-complementary sequences of each other (FIGS. 5C and 5D). The primer span, therefore, can be shortened to within 30 bases. Mismatches at the 5′end and/or the middle portion of the primers were introduced to reduce annealing of primers to each other and potential dimer formation (FIG. 5D). Second, TD-PCR is carried out using several pairs of primers, each slightly shifted within the coding sequence in comparison to the others (FIG. 5C and Table 1). We designed 6 pairs of TD-PCR primers located within exon 14 and 1 within intron 14 with a primer span ranged from 21-26 bases. ITD mutants with duplication length of less than the primer span are unlikely amplifiable even by this newly designed TD-PCR assay. ITD mutants with long duplication segments are expected to be amplified by 2 or more adjacent primer pairs. The size of amplicons derived from an individual ITD mutant should be the same or 1-5 base difference depending on the primer span of the primer pairs used to amplify the ITD mutant (FIG. 6). Amplification by two or more adjacent primer pairs with amplicons of the same size or few expected base difference indicates duplication specific amplification from an ITD mutant and were not observed in negative control samples.

TABLE 1
Primers for revised TD-PCR

| Primer | | |
|---|---|---|
| pairs | Primer | sequences |
| TD1 | TD1F | 5′-GACcagcatttcGtttccattgga-3′ (SEQ ID NO: 1) |
| | TD1R | 5′-AGGaatggaaaaCaaatgctgcag-3′ (SEQ ID NO: 2) |
| TD3 | TD3F | 5′-GtcaaatgggTgtttccaaga-3′ (SEQ ID NO: 3) |
| | TD3R | 5′-ActtggaaTctcccatttgag-3′ (SEQ ID NO: 4) |
| TD4 | TD4F | 5′-CatctcTaaAgggagtttcca-3′ (SEQ ID NO: 5) |
| | TD4R | 5′-AggaaactcccaAAtgagatc-3′ (SEQ ID NO: 6) |
| TD5 | TD5F | 5′-CTCATcttctTcgttgatttcagag-3′ (SEQ ID NO: 7) |
| | TD5R | 5′-GAGACaaatcTacgtagaagtactc-3′ (SEQ ID NO: 8) |
| TD6 | TD6F | 5′-GAGTCataatgTgtacttctacgttg-3′ (SEQ ID NO: 9) |
| | TD6R | 5′-GTTGCtagaagAactcattatctgag-3′(SEQ ID NO: 10) |
| TD7 | TD7F | 5′-ACTccggctcctcTgataatgag-3′ (SEQ ID NO: 11) |
| | TD7R | 5′-GAGattatctgTggagccggtca-3′ (SEQ ID NO: 12) |
| TD9 | TD9F | 5′-GagctacagTtggtacaggtg-3′ (SEQ ID NO: 13) |
| | TD9R | 5′-GacctgtaccTtctgtagctg-3′ (SEQ ID NO: 14) |

*Capitalized nucleotides: altered nucleotides to introduce mismatches

To test the limit of detection of TD-PCR, the present inventors selected a bone marrow specimen with an ITD mutant migrating at 456 bases by standard PCR. The bone marrow is comprised almost entirely of blasts and immature myeloid cells. Sanger sequencing of amplicons from standard PCR and TD-PCR confirmed an ITD of 129 bases extending from the 11th nucleotide of exon 14 (c.1715) to the 6th nucleotide of intron 14 (c.1882+6) and containing the primer sequences for primers pairs TD3, 4, 5, 6, 7 and 9. Amplicons of expected sizes were detected by each primer pairs (FIG. 6).

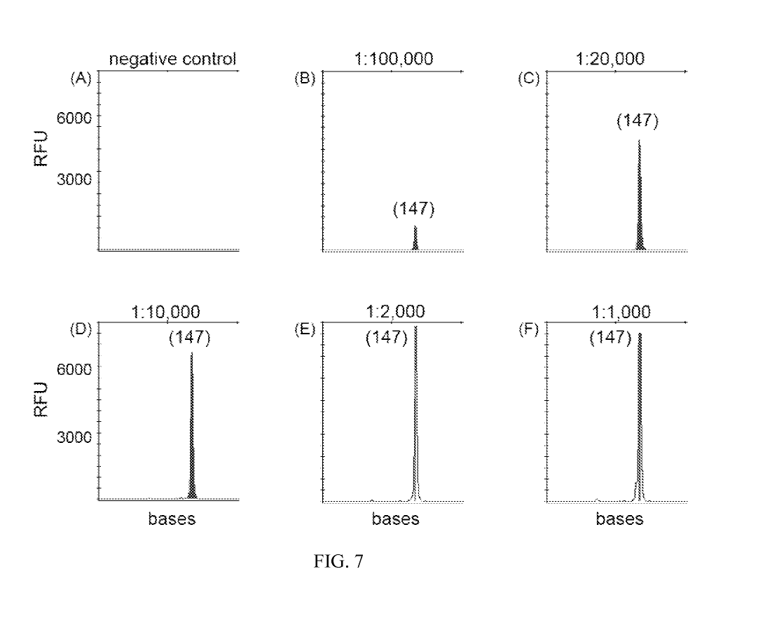

The specimen was serially diluted with an ITD negative specimen. TD-PCR was conducted in replicates with 250 ng DNA (equivalent to approximately 40,000 cells assuming each cell contains 6-7 pg DNA) in each reaction. The expected amplicons were detected 1 of 1 reaction at a dilution of 10⁻³, 5×10⁻⁴ and 10⁻⁴ dilution, 3-4 of 4 replicates at 5×10⁻⁵ dilution (equivalent to 2 mutant cells per reaction), and 1 of 4 replicates at 10⁻⁵ dilution (equivalent to 0.4 mutant cells per reaction) for primer pairs TD3, 5, 7 and 9 (FIG. 7). Amplicons was detected in 2 of 4 replicates at 5×10⁻⁵ dilution and 0 of 4 replicates at 10⁻⁵ dilution for TD4 and TD6. TD-PCR assay was also tested with a reduced amount of DNA (50 ng, equivalent to approximately 8,000 cells) at a dilution of 5×10⁻⁵ (equivalent to 0.4 mutant cells per reaction). The expected amplicon was also detected in 4 to 6 of 20 replicates for TD3, 5, 7 and 9. The peak heights of amplicons from TD3 and TD7 are relatively higher than those from the other primer pairs. The results indicate that TD-PCR is a very sensitive assay for detection minor clones and monitoring MRD of ITD mutations, capable of detecting a single mutant molecule.

TD-PCR detects up to 70% ITD mutants. ITD mutants from 58 newly diagnosed or frank relapsed FLT3/ITD AML patients were tested by TD-PCR using 50 ng DNA for each reaction of 7 primers pairs. Standard PCR assay has shown one, two and three ITD mutants in 53, 4 and 1 specimens, respectively (totally 64 dominant mutants detectable by standard PCR). Amplicons corresponding to the dominant ITD mutants were detected by only one primer pairs in 4 ITD mutants (6%) and by at least 2 primer pairs in 44 ITD mutants (69%). The estimated sizes of duplication segments of these 48 ITD mutants ranged from 36 to more than 170 bases as compared to 18-42 bases for those 16 ITD mutants with no corresponding amplicons detected by TD-PCR. As expected, ITD mutants with longer duplication trend to be detected by more pairs of primers. Forty-one of the 64 mutants (64%) can be amplified by both TD3 and TD7 or by either TD3 or TD7 primer pairs. The results showed that up to 70% of overall ITD mutants and most ITD mutants with a duplication segment of more than 40-50 bases could be amplified by TD-PCR.

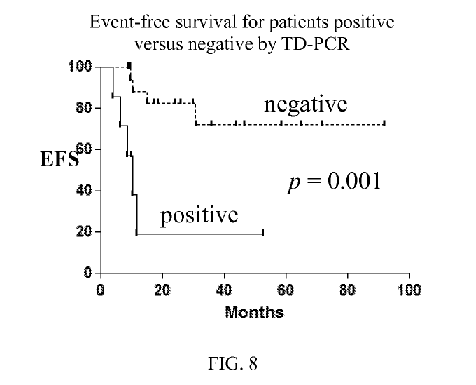

Application of TD-PCR in Minimal residual disease detection after hematopoietic cell transplantation. To investigate the potential clinical utility of TD-PCR, a chart review at the institution was performed and it was found that between December 2004 and May 2012 there were 62 patients diagnosed with FLT3/ITD AML who subsequently underwent allogeneic bone marrow transplant while in morphologic remission. Of those 62 patients, 50 were alive and in remission 60 days after the transplant. DNA samples prepared from bone marrow collected on or around Day 60 following the transplant were available for 36 of these 50 patients. In all 36 cases, the FLT3/ITD mutation was undetectable using the standard PCR assay. Based on the size of the duplications, the TD-PCR assay was informative for 26 of these 36 (72%). Of the 26 patients whose TD-PCR results were informative, seven (27%) had positive TD-PCRs on their day 60 bone marrow specimens. Of these seven patients with positive post-transplant TD-PCRs, five (71%) have relapsed to date, while two still remain in remission (one of whom is on sorafenib maintenance). Nineteen of the 26 (73%) had negative TD-PCRs on their day 60 bone marrow specimens. Of these 19 patients, only two (11%) have relapsed thus far, and both relapses were FLT3 wild type. TD-PCR is thus highly predictive (p=0.003) for relapse risk for FLT3/ITD AML following allogeneic transplant (FIG. 8). It is possible that this technique will identify patients who might benefit from post-transplant therapy such as FLT3 inhibition or donor lymphocyte infusion. The use of TD-PCR prospectively in an ongoing clinical trial using post-transplant sorafenib in FLT3/ITD AML patients is currently being evaluated.

Accordingly, this revised TD-PCR can be applied to clinical molecular diagnostics laboratories to guide post-transplant FLT3 inhibitor therapy and/or donor lymphocyte infusion in approximately 70% patients with FLT3/ITD mutation.

### DISCUSSION

ITD mutations of the FLT3 gene have been associated with a poor prognosis in AML. Detection of ITD mutations at diagnosis is now routine clinical practice. Although detection of minimal residual AML with ITD mutations is theoretically important, the current standard PCR assay for simultaneous detection of ITD and D835 mutations for newly diagnosed AML is not sensitive enough for monitoring MRD. In this study, the present inventors show that TD-PCR is a sensitive strategy to detect ITD mutation at a level of only a few molecules. In contrast to the current commercially available standard PCR assay, no amplification and, thus, no competition from the wild-type allele results in higher sensitivity in the TD-PCR assay.

Improved sensitivity can also be obtained by real-time PCR using mutant-specific primers or probes (8). However, each mutant needs a mutant-specific primer/probe designed from the junctional sequence; due to the constraints of the sequence at the junction, this may not be possible for every case. An added complication is that relatively low allelic burden mutants (as identified by standard PCR) may not be obtainable for direct sequencing since the wild type DNA is present in great excess. The present inventors have successfully isolated ITD mutants of less than 1% allelic frequency by using the molecular fraction collecting tool that the constructed (13, 14). The procedure, however, is cumbersome. In addition, it is laborious but mandatory to test the analytic sensitivity and specificity for each clone-specific PCR assay to have a valid clinical test. The single TD-PCR assay need only be validated once for clinical testing. ITD mutants at relapse occasionally differ from the mutant at diagnosis (3, 6, 15). These newly emerging ITD-positive mutants will not be detected by a real-time assay using mutant-specific primers/probes designed for MRD detection of a specific ITD mutant.

The TD-PCR assay can potentially be used to monitor MRD in approximately 20% of AML patients with ITD mutations. By designing additional primers within exon 14, more ITD mutants could be monitored by the TD-PCR assay. Our data confirmed the ability to capture more mutants with additional primer sets within exon 14. The sensitivity test can be performed universally using a single ITD-positive clone with a duplication extending from near the 5′ end of exon 14 to exon 15 or using a single long synthesized oligonucleotide.

Although TD-PCR is ultrasensitive as compared to the standard PCR or delta-PCR assays, short ITD mutations (less than about 40-50 bases) cannot be detected by TD-PCR since approximately 20 bases are needed for each primer. Delta-PCR, although not as sensitive as the real-time PCR assay or the TD-PCR assay, can provide broader screening of ITD mutations and monitoring of the emergence of a new ITD mutant. Combining delta-PCR and TD-PCR may provide the broadest and most sensitive monitor of MRD.

The initial design of TD-PCR assay detected ITD mutants with extremely low allelic burden in two cases that were reported as ITD-negative by standard PCR. Like most cases with ITD-negative AML cases, there was no serial follow-up of the FLT3 gene mutation in these two cases. Therefore, the clinical significance of such low allelic burden in these two cases is not known. In a previous report, the present inventors have shown that minor ITD clones with an allele burden of less than 1% at diagnosis became the dominant clone later in the disease course (11). It may be worthwhile to test all AML cases with TD-PCR serially and follow up those cases with initial extremely low mutant allelic burden.

In the subsequent studies, we have improved the design of TD-PCR to be applicable to approximately 70% FLT3/ITD AMLs. The assay was applied to detect MRD in a cohort of patients with hematopoietic cell transplantation and showed that detection of MDR by TD-PCR is prognostic of relapse. The assay was also used to detect ITD in normal bone marrows and to amplify and characterize the sequences of the minor ITD clones present in FLT3/ITD AML patients, patients with no ITD mutants detected by standard PCR assay and ITD positive cell lines (data not shown).

In summary, the present inventors have shown a proof-of-principle design of TD-PCR with an extremely high analytic sensitivity: a single molecule per reaction. TD-PCR assay can be applied in clinical molecular diagnostics laboratories for monitoring MRD of FLT3/ITD AML to guide post-transplant FLT3 inhibitor therapy and/or donor lymphocyte infusion and to identify AML patients with an extremely low allelic burden of ITD mutations at initial diagnosis. This approach can also be generalized and should be useful in any cancer, premalignant, infectious or other condition involving tandem duplications.

### REFERENCES

1. Blume-Jensen P, Hunter T. Oncogenic kinase signalling. Nature. 2001; 411:355-365.
2. Turner A M, Lin N L, Issarachai S, et al. FLT3 receptor expression on the surface of normal and malignant human hematopoietic cells. Blood. 1996; 88:3383-3390.
3. Kottaridis P D, Gale R E, Frew M E, et al. The presence of a FLT3 internal tandem duplication in patients with acute myeloid leukemia (AML) adds important prognostic information to cytogenetic risk group and response to the first cycle of chemotherapy: analysis of 854 patients from the United Kingdom Medical Research Council AML 10 and 12 trials. Blood. 2001; 98:1752-1759.
4. Meshinchi S, Woods W G, Stirewalt D L, et al. Prevalence and prognostic significance of Flt3 internal tandem duplication in pediatric acute myeloid leukemia. Blood. 2001; 97:89-94.
5. Thiede C, Steudel C, Mohr B, et al. Analysis of FLT3-activating mutations in 979 patients with acute myelogenous leukemia: association with FAB subtypes and identification of subgroups with poor prognosis. Blood. 2002; 99:4326-4335.
6. Schnittger S, Schoch C, Dugas M, et al. Analysis of FLT3 length mutations in 1003 patients with acute myeloid leukemia: correlation to cytogenetics, FAB subtype, and prognosis in the AMLCG study and usefulness as a marker for the detection of minimal residual disease. Blood. 2002; 100:59-66.
7. Schnittger S, Schoch C, Kern W, et al. FLT3 length mutations as marker for follow-up studies in acute myeloid leukaemia. Acta Haematol. 2004; 112:68-78.
8. Stirewalt D L, Willman C L, Radich J P. Quantitative, real-time polymerase chain reactions for FLT3 internal tandem duplications are highly sensitive and specific. Leuk Res. 2001; 25:1085-1088.
9. Nakao M, Yokota S, Iwai T, et al. Internal tandem duplication of the flt3 gene found in acute myeloid leukemia. Leukemia. 1996; 10:1911-1918.
10. Murphy K M, Levis M, Hafez M J, et al. Detection of FLT3 internal tandem duplication and D835 mutations by a multiplex polymerase chain reaction and capillary electrophoresis assay. J Mol Diagn. 2003; 5:96-102.
11. Beierl K, Tseng L H, Beierl R, et al. Detection of minor clones with internal tandem duplication mutations of FLT3 gene in acute myeloid leukemia using delta-PCR. Diagn Mol Pathol. 2012 (in press)
12. Lam K W, Jeffreys A J. Processes of de novo duplication of human alpha-globin genes. Pros Natl Acad Sci USA. 2007; 104:10950-10955.
13. Lin M T, Tseng L H, Rich R G, et al. Δ-PCR, a simple method to detect translocations and insertion/deletion mutations. J Mol Diagn. 2011; 13:85-92.
14. Lin M T, Rich R G, Shipley R F, et al. A molecular fraction collecting tool for the ABI 310 automated sequencer. J Mol Diagn. 2007; 9:598-603.
15. Shih L Y, Huang C F, Wu J H, et al. Internal tandem duplication of FLT3 in relapsed acute myeloid leukemia: a comparative analysis of bone marrow samples from 108 adult patients at diagnosis and relapse. Blood. 2002; 100:2387-2392.

1. A method for detecting an internal tandem duplication in the genome of a cell comprising the steps of:
a. providing a DNA sample isolated from the cell;
b. contacting the DNA sample with a forward primer that hybridizes within the tandem duplicated sequence;
c. contacting the DNA sample with a reverse primer upstream of the forward primer that hybridizes within the tandem duplicated sequence;
d. amplifying the region between the two primers; and
e. identifying the cell as containing an internal tandem duplication if an amplification product of the expected size is detected.

2. The method of claim 1, further comprising contacting the DNA sample with a second reverse primer upstream of the first reverse primer, wherein the amplification step produces two amplification products of two different sizes.

3. A method for detecting an internal tandem duplication in the genome of a cell comprising the steps of:
a. providing a DNA sample isolated from the cell;
b. contacting the DNA sample with a first primer set comprising:
i. a forward primer that hybridizes within the tandem duplicated sequence;
ii. a reverse primer upstream of the forward primer that hybridizes within the tandem duplicated sequence;
c. amplifying the region between the two primers; and
d. identifying the cell as containing an internal tandem duplication if an amplification product of the expected size is detected.

4. The method of claim 3, wherein the first primer set further comprises a second reverse primer upstream of the first reverse primer, wherein the amplification step produces two amplification products of two different sizes.

5. The method of claim 3, further comprising contacting the DNA sample with a second primer set comprising primers that hybridize to a different portion of the tandem duplicated sequence.

6. The method of claim 3, further comprising contacting the DNA sample with a second primer set comprising primers that hybridize to a sequence of the tandem duplicated sequence that overlaps with the first primer set.

7. A method for detecting an internal tandem duplication in the genome of a cell comprising the step of performing a polymerase chain reaction on DNA isolated from the cell using outward facing primers that hybridize within the tandem duplicated sequence.

8. A method for detecting an internal tandem duplication in a sample from a subject comprising the steps of:
a. providing a sample obtained from a subject;
b. contacting the sample with a forward primer that hybridizes within the tandem duplicated sequence;
c. contacting the sample with a reverse primer upstream of the forward primer that hybridizes within the tandem duplicated sequence;
d. amplifying the region between the two primers; and
e. identifying the sample as containing an internal tandem duplication if an amplification product of the expected size is detected.

9. The method of claim 8, further comprising contacting the sample with a second reverse primer upstream of the first reverse primer, wherein the amplification step produces two amplification products of two different sizes.

10. The method of claim 8, wherein the sample is a blood, plasma or serum sample.

11. The method of claim 8, wherein the subject is a human.

12. A kit for detecting internal tandem duplications comprising:
a. a forward primer that hybridizes with a tandem duplicated sequence;
b. a reverse primer that binds upstream of the forward primer that hybridizes within the tandem duplicated sequence; and
c. reagents for conducting an amplification reaction.

13. The kit of claim 12, further comprising a second reverse primer that binds upstream of the first reverse primer of element (b).
